(19) United States
(12) Patent Application Publication
(10) Pub. No.: US 2026/0151074 A1
(43) Pub. Date: Jun. 4, 2026

(54) SYSTEMS AND METHODS FOR USING NEURAL OBJECTIVE FUNCTIONS FOR CLOSED-LOOP OPTIMIZATION

(71) Applicant: Dandelion Science Corp., New York, NY (US)

(72) Inventors: Adam HANINA, Vandoeuvres (CH); David KLEIN, Los Altos, CA (US); Katerina MALAKHOVA, Highland Mills, NY (US); Jesse LIVEZEY, Jersey City, NJ (US)

(21) Appl. No.: 19/147,589

(22) PCT Filed: Jan. 12, 2024
(86) PCT No.: PCT/US2024/011453

§ 371 Date: Jul. 11, 2025

Related U.S. Application Data
(60) Provisional application No. 63/479,911, filed on Jan. 13, 2023.

(51) Int. Cl. A61B 5/378; A61B 5/00

(52) U.S. Cl. CPC A61B 5/378; A61B 5/0042; A61B 5/4836

(57) ABSTRACT

Systems and methods are disclosed that represent various advancements in neuroscience. For instance, systems and methods are disclosed for optimizing the processing of sensory information, refining diagnostic approaches for neurological disorders, and introducing novel methods for controlling brain network activity.

### CROSS-REFERENCE TO RELATED APPLICATIONS

This application is a US National Stage filing under 35 U.S.C. § 371 of International Application No. PCT/US2024/011453, filed Jan. 12, 2024, which claims priority to U.S. Provisional Application No. 63/479,911, filed on Jan. 13, 2023, the entireties of which are incorporated herein by reference.

### TECHNICAL FIELD

The present disclosure relates generally to the field of neuroscience and neuroengineering and, more specifically, to systems and methods that leverage novel methodologies that integrate computational neuroscience, signal processing, and machine learning.

### BACKGROUND

Sensory systems play a critical role in facilitating the flow of information to non-sensory cortical areas. However, direct optimization in downstream regions of sensory systems may be hindered by sparse or weak activity of neural pathways. Additionally, diagnostic tools for complex neurological disorders, such as visual neglect and autism, may lack sensitivity and the ability to accommodate the individualized nature of these conditions. Furthermore, discovering effective sensory inputs for influencing brain network activity may be hindered by impractical and/or inefficient searching through vast input spaces within limited timeframes.

The present disclosure is accordingly directed to innovative approaches in neuroscience for optimizing sensory information, diagnosing neurological disorders, and affecting brain network activity. These techniques collectively offer transformative advancements with potential applications in research and clinical interventions. The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for refining the processing of sensory information, the results of which may be utilized in various downstream applications.

In one aspect, a computer-implemented method for performing iterative adjustments to sensory stimuli in a closed-loop optimization system is provided. The computer-implemented may include: receiving, at a computing device, brain activity data associated with a stimulus presented to a subject; preprocessing, using a processor associated with the computing device, the brain activity data; performing, using the processor, a source localization technique on the preprocessed brain activity data to identify one or more voxel locations where electrical activity contained in the brain activity data is projected to originate; grouping, subsequent to performance of the source localization technique and based on the preprocessed brain activity data, a first subset of the one or more voxel locations together, wherein the first subset includes is associated with sensory areas of interest; grouping, subsequent to performance of the source localization technique and based on the preprocessed brain activity data, a second subset of the one or more voxel locations together, wherein the second subset is associated with non-sensory areas of interest; determining, using the processor, a first objective function based on localized neural activity identified within the first subset of the one or more voxel locations and determining a second objective function based on the localized neural activity identified within the second subset of the one or more voxel locations; determining, using the processor, whether the second objective function is equivalent to or exceeds a threshold minimum value; averaging, responsive to determining that the second objective function is equivalent to or exceeds the threshold minimum value, an amount of the second objective function over the threshold minimum value with the first objective function to produce an averaged objective function; and utilizing the averaged objective function to refine one or more parameters of the stimulus in a subsequent iteration of the closed-loop optimization system.

In another aspect, a system for performing iterative adjustments to sensory stimuli is provided. The system may include: one or more processors; one or more computer readable media storing instructions that are executable by the one or more processors to perform operations for: receiving brain activity data associated with a stimulus presented to a subject; preprocessing the brain activity data; performing a source localization technique on the preprocessed brain activity data to identify one or more voxel locations where electrical activity contained in the brain activity data is projected to originate; grouping, subsequent to performance of the source localization technique and based on the preprocessed brain activity data, a first subset of the one or more voxel locations together, wherein the first subset includes is associated with sensory areas of interest; grouping, subsequent to performance of the source localization technique and based on the preprocessed brain activity data, a second subset of the one or more voxel locations together, wherein the second subset is associated with non-sensory areas of interest; determining a first objective function based on localized neural activity identified within the first subset of the one or more voxel locations and determining a second objective function based on the localized neural activity identified within the second subset of the one or more voxel locations; determining whether the second objective function is equivalent to or exceeds a threshold minimum value; averaging, responsive to determining that the second objective function is equivalent to or exceeds the threshold minimum value, an amount of the second objective function over the threshold minimum value with the first objective function to produce an averaged objective function; and utilizing the averaged objective function to refine one or more parameters of the stimulus in a subsequent iteration of a closed-loop optimization function of the system.

In yet another aspect, a non-transitory computer-readable medium storing computer-executable instructions is provided. The non-transitory computer-readable medium stores computer-executable instructions which, when executed by a system, may cause the system to perform operations including: receiving, at a computing device associated with the system, brain activity data associated with a stimulus presented to a subject; preprocessing, using a processor associated with the computing device, the brain activity data; performing, using the processor, a source localization technique on the preprocessed brain activity data to identify one or more voxel locations where electrical activity contained in the brain activity data is projected to originate; grouping, subsequent to performance of the source localization technique and based on the preprocessed brain activity data, a first subset of the one or more voxel locations together, wherein the first subset includes is associated with sensory areas of interest; grouping, subsequent to performance of the source localization technique and based on the preprocessed brain activity data, a second subset of the one or more voxel locations together, wherein the second subset is associated with non-sensory areas of interest; determining, using the processor, a first objective function based on localized neural activity identified within the first subset of the one or more voxel locations and determining a second objective function based on the localized neural activity identified within the second subset of the one or more voxel locations; determining, using the processor, whether the second objective function is equivalent to or exceeds a threshold minimum value; averaging, responsive to determining that the second objective function is equivalent to or exceeds the threshold minimum value, an amount of the second objective function over the threshold minimum value with the first objective function to produce an averaged objective function; and utilizing the averaged objective function to refine one or more parameters of the stimulus in a subsequent iteration of a closed-loop optimization system.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1A depicts an exemplary computer system for executing the methods described herein.

FIG. 1B depicts an exemplary software platform for executing the methods described herein.

FIG. 2 depicts an exemplary workflow for performing closed-loop optimization to enhance non-sensory brain activity, according to one or more embodiments of the present disclosure.

FIG. 3 depicts an exemplary workflow for diagnosing neurological disorders with visual system function based diagnostics, according to one or more embodiments of the present disclosure.

FIG. 4 depicts an exemplary workflow for a first process involved in utilizing spontaneous brain activity to constrain sensory inputs used to control brain activity, according to one or more embodiments of the present disclosure.

FIG. 5 depicts an exemplary workflow for a second process involved in utilizing spontaneous brain activity to constrain sensory inputs used to control brain activity, according to one or more embodiments of the present disclosure.

FIG. 6 depicts an exemplary workflow for utilizing short time analysis windows and hyperscanning for neural SNR maximization for closed-loop optimization, according to one or more embodiments of the present disclosure.

FIG. 7 depicts another exemplary workflow for utilizing short time analysis windows and hyperscanning for neural SNR maximization for closed-loop optimization, according to one or more embodiments of the present disclosure.

FIG. 8 depicts another exemplary workflow for utilizing short time analysis windows and hyperscanning for neural SNR maximization for closed-loop optimization, according to one or more embodiments of the present disclosure

FIG. 9 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Visual perception, the ability to see and understand the world around us, is important to the quality of life and independence of most humans. In healthy visual processing, light activates photoreceptive cells in the retina of the eye, which innervate an expansive visual processing network comprising over half of the brain's cortex. This network may in turn innervate other areas of the brain. Unfortunately, disease or injury to either the eyes, optic nerves, or in nonsensory regions of the brain, can severely compromise visual functioning or other neurological function. Such disorders are a global problem affecting all demographics, but are especially prevalent amongst older adults. Pertinent examples include age-related macular degeneration (AMD), diabetic retinopathy, glaucoma, myopia, cortical visual impairment, visual spatial neglect, stroke, Parkinsonian syndrome, epilepsy, multiple sclerosis, deafness, balance disorders, tumor-induced dysfunction, palsies, autism, visual neglect, and even psychiatric disorders such as depression and schizophrenia, to name a few.

Techniques described herein assess and treat such sensory and non-sensory disorders via sensory stimulation. But the ability to treat such disorders, as well as the fields of research investigating novel interventions and treatment pathways, require reliable and valid measurements of visual processing. It is important for clinicians to understand how a patient's sensory processing capacity is changing as a disease progresses or an injury recovers, and for researchers to objectively measure how different interventions might affect these changes in sensory function. The neuroscientific principles governing visual and other sensory perceptual processing are dynamic and not fully understood. Indeed, imaging approaches to measure the degree and location of damage in the eye or brain often correlate poorly with reported visual function. To account for this, gold standard clinical approaches typically rely on behavioral data and self-reporting. For example, common measures of visual function include the use of Snellen charts (e.g., charts comprised of lines of progressively smaller text to evaluate the smallest print size which patients can reliably report), evaluations of reading speed, Amsler grids (e.g., in which patients report distortions or missing areas while viewing a grid pattern); and automated visual field tests (e.g., in which patients report whether visual stimuli presented at different parts of their visual field can be seen or not). Such tests typically rely on thresholding (e.g., in which it is inquired whether a stimulus is visible or not) and thus might not be sensitive to subtle changes in visual function. Further, behavioral assessments require patients to be able to make a behavioral response, which might not be possible for some patient groups (e.g., infants, young children, traumatic brain injury and stroke patients, and patients with intellectual disabilities).

To address these limitations of behavioral measures, testing may be performed to harness neuroimaging to assess neurological function. These may involve recording neural responses to visual or other sensory stimuli using electroencephalography (EEG) and/or magnetoencephalography (MEG). EEG is a relatively affordable, widely available neuroimaging technique that measures the electrical activity generated by neurons in the brain's cortex using electrodes placed at the scalp. One set of techniques for the visual system, which may be termed visual evoked potential (VEP) measures of visual acuity, measure neural responses to a sweep of simple pattern-reversing grating stimuli across a range of spatial frequencies. These techniques may harness steady-state visual evoked potentials (SSVEPs) to measure the threshold highest granularity of visual information that a patient can perceive. SSVEPs are oscillatory neural responses to flickering visual stimuli. When a visual stimulus is flickered at a set frequency, visual cortical neurons involved in processing that stimulus respond at the same frequency, a response which can be detected in the EEG signal. Transforming the EEG signal from the time-domain to the frequency domain, using methods such as fast Fourier transforms (FFTs) or wavelet decomposition, allows for objective analysis of how strongly the flicker frequency is represented in the signal (termed the SSVEP amplitude). In SSVEP protocols for evaluating visual acuity, these SSVEP amplitudes may be used to evaluate the threshold spatial frequency for which SSVEPs can no longer be detected. Spatial frequency is the rate at which information changes over space; thus the more fine and detailed visual information is (i.e., very small checks in a textured grating) the higher the spatial frequency. In these techniques, SSVEPs may be elicited by reversing the black and white elements of a textured grating at a set rate, thus when the spatial frequency becomes too high to resolve, the pattern may appear to be a uniform gray, and no longer evokes an SSVEP. A draw-back of this method is that, much like many behavioral tests, it relies on a threshold, and thus does not allow for any exploration of subtle changes in visual function or response profiles.

Treatment of nonsensory regions of the brain using sensory stimuli may also be challenging, as it may be difficult to detect the effect sensory stimuli has on nonsensory regions of the brain. Distinguishing the faint signal of such downstream effects may be difficult due to large amounts of noise picked up by detection devices (e.g., EEG or MEG). Determining effective treatments is complicated by the large space of possible inputs (e.g., visual stimulatory images), only a small subset of which may have a therapeutic effect. And particular ailments may have idiosyncratic neurological signatures that complicate both the determination of effective sensory inputs and the determination of disease state and progression.

Accordingly, the present disclosure contemplates pioneering advancements in neuroscience, proposing innovative techniques for optimizing the processing of sensory information, refining diagnostic approaches for neurological disorders, and introducing novel methods for controlling brain network activity. These innovations collectively offer transformative applications with potential implications for research, clinical diagnostics, and therapeutic interventions in the field of neuroengineering.

In an aspect, the collective concepts presented in this disclosure offer concrete and tangible applications in neuroscience and neuroengineering. Additionally, these concepts represent improvements in computer technology by introducing innovative applications at the intersection of neuroscience and computing. For instance, the closed-loop optimization techniques (e.g., those techniques by which parameters are optimized in an iterative way, for instance by an algorithm, until optimal parameters are identified) applied to sensory systems leverage advanced algorithms and real-time observation using EEG/MEG devices, showcasing a novel integration of computational methodologies to enhance sensory information processing. The high-fidelity functional mapping of the visual system using EEG and/or MEG demonstrates an application of advanced signal processing techniques, providing a more nuanced understanding of neurological disorders. Additionally, the methods of discovering and constraining sensory inputs for controlling brain network activity may involve machine learning algorithms and computational modeling, showcasing a clear improvement in the computational tools used for brain research. Furthermore, because the concepts described herein involve intricate computational processes that are reliant on the capabilities of external computing systems and specialized equipment, they cannot be performed in the human mind. While the human mind plays a role in designing, interpreting, and utilizing these methodologies, the actual execution of these advanced techniques goes beyond the inherent computational capacity of the human brain.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

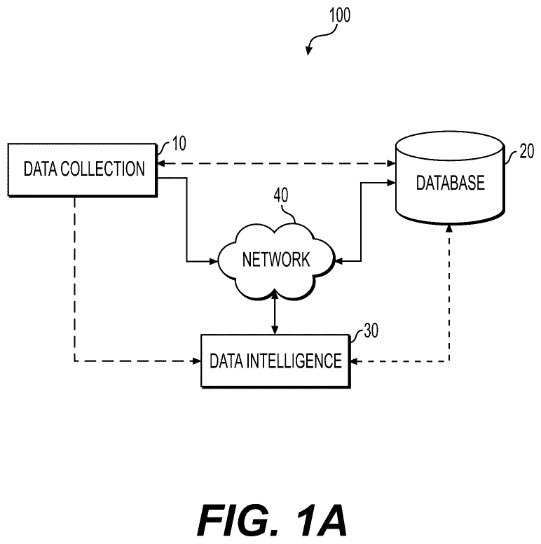

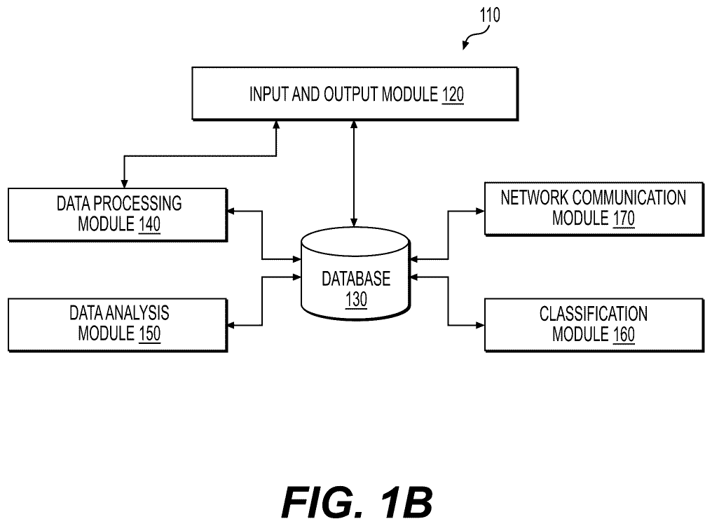

FIG. 1A depicts an exemplary system by which the methods described herein may be executed. Exemplary system 100 includes a data collection component 10, a database 20, and device data intelligence component 30, operably connected to each other via network 40. Alternatively, or additionally, one or more of the components may be connected with another component locally without reliance on network connection; e.g., through a wired connection.

As disclosed herein, data collection component 10 may include a device or machine with which electrical activity in the brain may be measured. In some embodiments, data collection component 10 may be an EEG machine that contains, or is configured to support, one or more electrodes, amplifiers, filters, analog to digital converters, etc., by which to conduct an EEG test. In some aspects, data collection component 10 may be a database that receives EEG test data from one or more other sources. In other aspects, data collection component 10 may be any other brain recording device or modality that may convey information about neural activity.

Data acquired by the data collection component 10 may be transferred to database 20 via network 40 or a local or network connection. In some embodiments, the collected data may be analyzed by data intelligence component 30, via network 40 or a local or network connection. FIG. 1B depicts exemplary functional modules that may be implemented to perform tasks of data intelligence component 30.

FIG. 1B depicts an exemplary computer system 110 for using techniques discussed herein, for example neural objective functions for closed-loop optimization.

Exemplary system 110 achieves the techniques discussed herein by implementing, on one or more computer devices, user input and output (I/O) module 120, memory or database 130, data processing module 140, data analysis module 150, classification module 160, network communication module 170, and any other functional modules that may be needed for carrying out a particular task (e.g., an error correction or compensation module, a data compression module, etc.). These modules may correspond to the modules of FIG. 1A. For example, database 130 may correspond to database 20, modules 140, 150, 160, and 170 may correspond to data intelligence 30, and the input aspect of module 120 may correspond to data collection 10. As disclosed herein, user I/O module 120 may further include an input sub-module, such as a keyboard, MEG, EEG, eye tracking data, and an output sub-module, such as a display (e.g., a printer, a television, a smartphone, a monitor, a virtual reality (VR) device, and/or a touchpad). In some embodiments, all functionalities may be performed by one computer system. In some embodiments, the functionalities are performed by more than one computer system. The various modules (e.g., for data processing, analysis, classification, communication, etc.) may be one or more processes executing in a distributed computing environment. For instance, in some embodiments, one or more components of the computer system 110 may be network accessible via cloud infrastructure. For example, the database 130 used to store data may be stored in one or more remote cloud servers. In this regard, the database may be one or more large storage buckets (e.g., cloud-based storage buckets such as simple storage service “S3” buckets, etc.) from which data may be retrieved on demand. As another example, data processing, analysis, and classification may be performed in cloud-based environments using services like cloud-based data processing platforms, serverless computing, cloud-based machine learning platforms, and the like.

Also disclosed herein, a particular task may be performed by implementing one or more functional modules. In particular, each of the enumerated modules itself may, in turn, include multiple sub-modules implementing one or more techniques discussed herein. For example, data processing module 140 may include a sub-module for data quality evaluation (e.g., for performing iterative refinement and validation), a sub-module for normalizing any assigned weights to ensure that the weights contribute proportionally to the overall response, a sub-module for performing interpolation or extrapolation, and the like.

In some embodiments, a user may use I/O module 120 to manipulate data that is available either on a local device or can be obtained via a network connection from a remote service device or another user device. For example, I/O module 120 may allow a user, e.g., via a keyboard, a mouse, or a touchpad, to perform data analysis via a graphical user interface (GUI). In some embodiments, a user may manipulate data via voice control. In some embodiments, user authentication may be required before a user is granted access to the data being requested. In some embodiments, user I/O module 120 may be used to manage various functional modules. For example, a user may request via user I/O module 120 input data while an existing data processing session is in process. A user may do so by selecting a menu option or type in a command discretely without interrupting the existing process. In another example, a user may utilize user I/O module 120 to set various thresholds, configure sample matching settings, and/or provide other instructions to computer system 110 that dictate how electrical signals in the brain are captured and/or monitored. As disclosed herein, a user may use any type of input to direct and control data processing and analysis via I/O module 120.

In some embodiments, system 110 further comprises a memory and/or database 130. In some embodiments, database 130 comprises a local database that may be accessed via user I/O module 120. In some embodiments, database 130 comprises a remote database that may be accessed by user I/O module 120 via network connection. In some embodiments, database 130 is a local database that stores data retrieved from another device (e.g., a user device or a server). In some embodiments, memory or database 130 may store data retrieved in real-time from internet searches. In some embodiments, database 130 may send data to and receive data from one or more of the other functional modules, including, but not limited to, a data collection module (not shown), data processing module 140, data analysis module 150, classification module 160, network communication module 170, and etc. In some embodiments, some or all real-sample data and/or synthetic sample data may be stored on database 130.

In some embodiments, database 130 may be a database local to the other functional modules. In some embodiments, database 130 may be a remote database that may be accessed by the other functional modules via wired or wireless network connection (e.g., via network communication module 170). In some embodiments, database 130 may include a local portion and a remote portion.

In some embodiments, system 110 comprises a data processing module 140. Data processing module 140 may receive the real-time data, from I/O module 120 or database 130. In some embodiments, data processing module 140 may perform standard data processing algorithms, such as one or more of noise reduction, signal enhancement, normalization, interpolation and/or extrapolation, etc. In some embodiments, data processing module 140 may be configured to process received and/or collected neural activity data associated with one or more subjects. In various embodiments, data processing module 140 may additionally create a training data set, on which one or more machine-learning models (e.g., for classification, clustering, scoring, etc.) may be trained.

In some embodiments, system 110 comprises a data analysis module 150. In some embodiments, data analysis module 150 includes identifying brain activity patterns associated with particular medical conditions, as described in connection with data processing module 140.

In some embodiments, system 110 comprises a classification module 160, which may embody a “machine-learning model” or “trained classifier.” As used herein, a “machine-learning model” or “trained classifier” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as k-nearest neighbors, linear regression, logistic regression, random forest, gradient boosted machine (GBM), deep learning, a deep neural network (e.g., recurrent neural network (RNN), convolutional neural network (CNN)) and/or any other suitable machine-learning technique that solves problems in the field of Natural Language Processing (NLP). Supervised, semi-supervised, and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

In an exemplary use case, a machine-learning model may be trained to analyze test data from a test subject whose specific neural activity with respect to a medical condition may be unknown and then subsequently identifying portions or characteristics of the test subject's brain that may be responsible for or may be resultant of the medical condition. In some embodiments, the one or more parameters may include a score (e.g., a binomial probability score that may be calculated based on logistic regression analysis). As disclosed herein, the binomial probability score may correspond to the likelihood of a subject having a certain medical condition, the likelihood of a portion of the subject's brain being active or inactive, the likelihood of a particular stimuli affecting a desired portion of the brain, etc. For example, a score of over a predefined threshold may indicate that a specific stimuli has effectively stimulated a non-sensory region of the brain.

As disclosed herein, network communication module 170 may be used to facilitate communications between a user device, one or more databases, and any other suitable system or device through a wired or wireless network connection. Any communication protocol/device may be used, including, without limitation, a modem, an Ethernet connection, a network card (wireless or wired), an infrared communication device, a wireless communication device, and/or a chipset (such as a Bluetooth ™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for processing/analyzing tumor fraction data may communicate with another user device with the same platform, a regular user device without the same platform (e.g., a regular smartphone), a remote server, a physical device of a remote IoT local network, a wearable device, a user device communicably connected to a remote server, and etc.

Techniques disclosed herein may be used in combination with those discussed in U.S. Pat. No. 10,736,526 and U.S. application Ser. No. 18/044,054, each of which are incorporated by reference herein in their entireties.

The functional modules described herein are provided by way of example. It will be understood that different functional modules may be combined to create different utilities. It will also be understood that additional functional modules or sub-modules may be created to implement a certain utility.

### Neural Objective Functions for Closed-Loop Optimization of Sensory System Throughput

Neuroscientific research has long sought ways to harness the potential of sensory stimuli to influence brain activity beyond traditional sensory areas, aiming to unlock broader cognitive enhancements or therapeutics. The challenge lies in the inherent limitations of conventional methods, which primarily focus on stimulating sensory regions and often face difficulties in effectively engaging non-sensory areas due to irregular, weak, or undetectable responses. More particularly, when attempting direct stimulation in non-sensory areas, the sparse nature of neural responses poses a significant obstacle because without immediate and informative feedback during the optimization process, it becomes challenging to discern the efficacy of stimuli and/or challenging to make iterative adjustments toward the desired objective. The limitations of these conventional approaches become apparent when attempting to apply closed-loop feedback in areas where neural activity may not manifest immediately or robustly.

The concepts described herein introduce an improvement in the optimization of sensory stimuli for enhancing non-sensory brain activity. By focusing on closed-loop optimization within sensory areas, the system may overcome the limitations encountered by conventional approaches. More particularly, through the use of neural objective functions, e.g., based on sensory cortical functionality metrics and data throughput metrics, the described concepts allow for a gradual and informed approach that ensures that stimuli are systematically optimized with informative feedback from sensory areas, which may guide the process until desired effects extend into non-sensory regions. The closed-loop system, incorporating EEG data, for example, and advanced optimization techniques (e.g., such as Bayesian Optimization), represents a substantial improvement over conventional methods in stimulating non-sensory brain areas for cognitive enhancement.

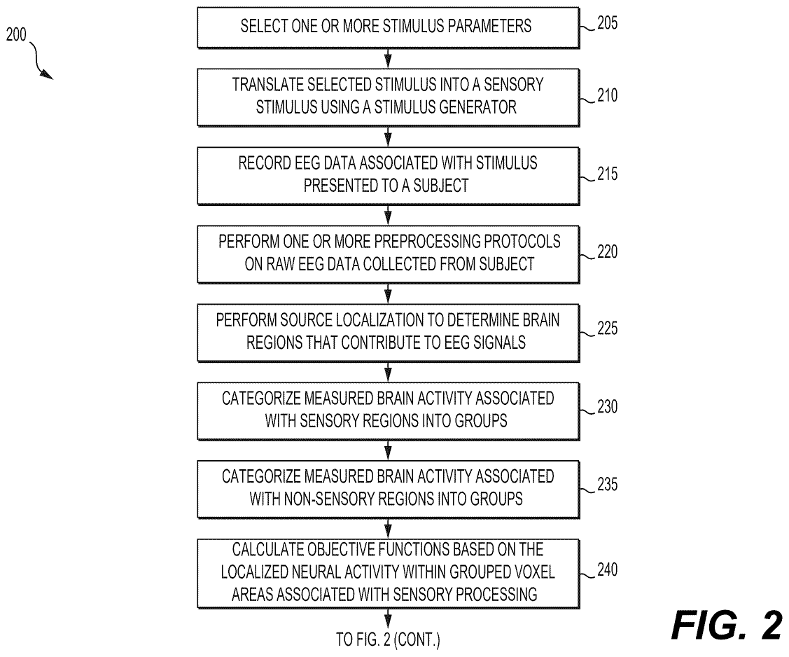

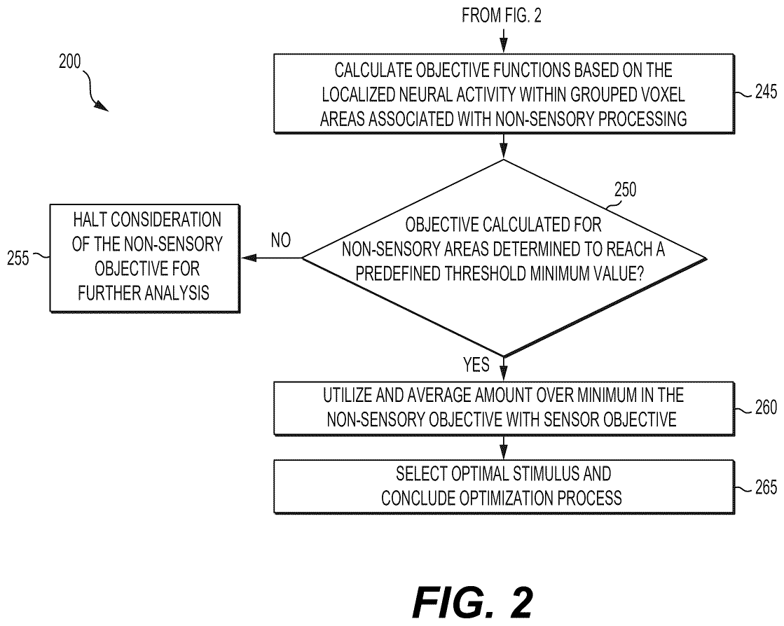

Referring now to FIG. 2, an exemplary workflow 200 is provided for performing closed-loop optimization to enhance non-sensory brain activity. Aspects of the exemplary workflow 200 may be performed in accordance with some or all components described in FIGS. 1A and 1B.

At step 205, the optimization process may begin with the selection of one or more stimulus parameters, which represent the characteristics of sensory stimuli to be presented to the participant. In an aspect, stimulus parameters refer to the various attributes and properties that define a sensory stimulus. These parameters may include, but are not limited to, visual attributes (e.g., color, contrast, motion, length, etc.), auditory features (e.g., frequency, intensity, duration, etc.), other sensor modalities, and/or any combination of the foregoing. In an aspect, the selection may be driven by the overall objective of the closed-loop optimization process. In this context, the objective may be to enhance certain non-sensory brain activity. Therefore, predetermined stimulus parameters are chosen based on their potential to influence neural responses in both sensory and non-sensory areas.

In an aspect, the optimization system may employ techniques such as Bayesian Optimization to iteratively explore a range of stimulus parameters. This exploration may involve selecting a set of parameters, presenting the corresponding stimulus, recording EEG responses, and using the collective data to inform the next set of parameters, as further described herein. Initial parameters may be selected based upon patient demographics such as age or sex, prior known generally effective parameters, predetermined parameters given the symptoms and/or medical condition of the patient, and/or based on initial EEG readings or brain images of the patient. The closed-loop nature of the system allows for adaptive adjustments to the stimulus parameters based on real-time feedback. For instance, if certain parameters prove to be more effective in eliciting responses, the optimization process may dynamically shift towards exploring similar parameter combinations. Furthermore, stimulus parameters might not be fixed, but rather may be refined iteratively through multiple optimization cycles, as further described herein.

At step 210, the chosen stimulus parameters may be translated into a sensory stimulus using a stimulus generator. This translation process ensures that the characteristics and features defined by the parameters are accurately represented in the generated stimulus. In an aspect, the nature of the sensory stimulus depends on the sensory modality under consideration. For example, if the study focuses on visual stimuli, the generated stimuli may be a visual pattern or sequence. Similarly, as another example, for auditory stimuli, the generated stimuli may involve a specific sequence of tones or sounds. In an aspect, the temporal and spatial aspects of the stimulus may be important. For instance, the duration, timing, and/or spatial properties (if applicable) may be determined based on the experimental requirements and the sensory systems being targeted.

In an aspect, a stimulus generator may be employed to create the sensory stimulus according to the defined parameters. The stimulus generator may be, for example, a computer program or one or more pieces of specialized stimulus-generating hardware. The stimulus generator may ensure precision and consistency in presenting stimuli across different iterations of the optimization process. In an aspect, the generated stimulus may be presented for a predetermined duration, ensuring that there is sufficient time for the participant's brain to respond. The stimulus may be repeated or presented in a specific sequence to capture variations in neural responses. In some circumstances, participants may be instructed to interact with or respond to the presented stimuli (e.g., in studies when exploring cognitive or motor response in addition to neural activity). In an aspect, if the optimization algorithm indicates a need for adjustments based on ongoing, for example, EEG data, the system may dynamically modify the stimulus parameters for the next iteration, as further described herein.

At step 215, the translated sensory stimulus may be presented to the subject and EEG data may be recorded. In an alternative technique, other brain imaging modalities may be used such as MEG. EEG is a non-invasive neuroimaging technique that records the electrical activity of the brain through electrodes placed on the scalp. It provides a real-time measure of neural activity, capturing the synchronized firing of neurons. Electrodes may be strategically placed on the subject's scalp according to a standardized or customized configuration based on the experimental design. In some aspects, the choice of electrode locations is often determined by the specific brain regions under investigation. The EEG system may record electrical potentials generated by the brain over time. The recorded signals represent the sum of postsynaptic potentials of neurons in the vicinity of each electrode. EEG offers high temporal resolution, allowing for the precise tracking of rapid changes in neural activity, making it particularly well-suited for capturing dynamic responses to sensory stimuli.

In an aspect, EEG data recording may be conducted in a continuous fashion throughout the presentation of the sensory stimuli. This ensures that the entire duration of the stimulus, as well as any post-stimulus effects, is captured for analysis.

Continuous monitoring of EEG signals may be important to identify artifacts that arise during recording. These artifacts may result from eye movements, muscle activity, external sources, etc., and their presence may impact the accuracy of the recorded neural signals.

At step 220, the data recorded during the EEG session may initially be in raw form (e.g., representing the electrical activity of the brain as a function of time) and various techniques may be employed to “clean” and preprocess this raw data to enhance the quality of the recorded signals, remove artifacts, and prepare the data for subsequent analysis. For instance, one or more filtering techniques may be applied to the raw EEG data to isolate the specific frequency bands of interest (e.g., by screening out frequencies that are too low or too high). As another example, various techniques (e.g., independent component analysis (ICA), etc.) may be employed to remove artifacts from the EEG data. In yet another example, a baseline correction process may be employed to adjust the EEG data to a common baseline to help in eliminating baseline drift and ensure subsequent analyses focus on changes in neural activity relative to a stable reference point. Additionally or alternatively to the foregoing, one or more other types of filtering and/or normalization techniques may be employed.

At step 225, a source localization process may be conducted to determine the anatomical locations or brain regions that contribute to the recorded EEG signals. This step may help determine the neural basis of observed responses and tailor stimuli for optimal impact on targeted brain areas (e.g., on the non-sensory regions). One or more models and/or algorithms, e.g., such as Low-Resolution Electromagnetic Tomography Analysis (LORETA), may be leveraged to perform source localization. In an aspect, source localization often results in the identification of voxel locations corresponding to potential neural sources. These voxels are three-dimensional points in the brain space where neural activity is determined to originate. In an aspect, the chosen source localization algorithm, e.g., LORETA, may define voxel activity by a function of EEG channel activities. For instance, each voxel may have a different defined function. Ultimately, at the end, instead of per-channel activity, “per-voxel” activity may be obtained.

At step 230, the measured brain activity may be categorized into distinct groups or clusters based on their spatial and/or functional characteristics. In an aspect, the brain's sensory processing involves hierarchical stages, especially in areas dedicated to processing specific sensory modalities, such as vision. For example, in the visual system, information travels through different cortical areas, each responsible for extracting and processing specific features. The sensory areas, like V1, V2, V4, MT (Medial Temporal Area), are identified based on their known roles in the hierarchical processing of sensor information. Each area is associated with distinct functions in processing visual stimuli. As alluded to above, a “voxel” represents a three-dimensional unit in which brain activity is associated and measured. In neuroimaging, such as through EEG source localization (as performed at step 225), the brain is divided into these small volumetric units with which to associate neural activity patterns.

In an aspect, the spatial coordinates of voxels within the identified sensory areas are considered. Voxels in a particular area may be grouped together based on their proximity and functional relevance to that specific stage in the sensory processing hierarchy. Grouping may also take into account the spatial-temporal connectivity patterns between voxels. The grouped voxels not only share spatial proximity, but also exhibit synchronized or functionally connected neural activity, reflecting their involvement in the same sensory processing stage. Once voxel groups corresponding to different sensory areas are established, objective functions may be calculated based on the localized activity across each group, as further described herein. Machine learning or deep learning models may be used to optimize stimulus according to these objective functions.

At step 235, measured brain activity in brain regions that are not primarily associated with sensory processing, i.e., the non-sensory regions, may be organized and categorized into distinct groups or clusters based on spatial and functional characteristics. In the context of this application, non-sensory areas correspond to those regions of the brain that are not primarily responsible for processing sensory inputs but may have downstream or higher-order functions.

Examples include frontal areas, centrotemporal areas, motor cortex, posterior parietal lobe, etc.

The spatial coordinates of voxels within the identified non-sensory areas may be grouped together based on their spatial proximity and functional relevance within that specific region. In an aspect, grouping may also take into account the spatial-temporal connectivity patterns between voxels. This ensures that the grouped voxels not only share spatial proximity but also exhibit functionally connected neural activity, reflecting their involvement in the same non-sensory processing regions. The grouping of voxels in non-sensory areas complements the earlier grouping in sensory areas. The objective is to understand and optimize neural activity in these non-sensory regions based on the overall goal of stimulating non-sensory brain regions through sensory inputs.

At step 240, objective functions may be calculated based on the localized neural activity within grouped voxel areas associated with sensory processing. In an aspect, the objective functions may provide quantitative measures of the neural responses in these sensory regions, thereby guiding the optimization process. In an aspect, the objective calculation may begin with measuring the localized neural activity within each voxel group. This may involve assessing the strength, patterns, or characteristics of neural responses in the specified sensory areas. In an aspect, one example of a metric used for objective calculation may be the average power of neural activity within each voxel group in non-sensory areas. This may involve quantifying the amplitude or intensity of neural oscillations in a given frequency band, thereby providing a measure of the overall activity. In an aspect, another example of a usable metric may be the slope of power across the sensory areas. This metric assesses how the power of neural activity changes when moving from one sensory area to another, thereby capturing the dynamics and progression of neural responses in the hierarchical sensory processing pathway. Other metrics not explicitly listed and/or described here, e.g., such as the degree of phase locking across different areas, may also be described and/or utilized.

At step 245, objective functions may be calculated based on the localized neural activity within grouped voxel areas associated with non-sensory areas of the brain. The objective functions provide quantitative measures of the neural responses in these non-sensory areas, further guiding the optimization process. Similar to step 240, objective calculation may begin with measuring the localized neural activity within each voxel group associated with non-sensory areas, which may involve assessing the strength, patterns, or characteristics of neural responses in the non-sensory areas. Metrics such as average power of neural activity, slope of power, and/or one or more other metrics, as previously described above, may be utilized. Additional metrics, such as the identification of peaks in the frequency spectrum, detection of phase locking oscillations in a frequency band across two different non-sensory areas (e.g., wherein phase locking implies a fixed time relationship between two waveforms, and in particular for sinusoidal components of the waveforms, they have a fixed phase relationship between the two), may also be utilized.

At step 250, a decision-making process may be employed to determine whether the objective calculated for non-sensory areas has reached a predefined threshold minimum value. Before researching this decision point, objective functions have been calculated based on the localized neural activity within grouped voxel areas associated with non-sensory regions of the brain. These objectives represent quantitative measures of the neural responses in non-sensory areas. In an aspect, a predefined minimum value may be set for the objective in the non-sensory areas. This minimum value may serve as a threshold that the calculated objective must meet and/or surpass for the optimization process to consider the non-sensory objectives as satisfactory.

In an aspect, a comparison may be made between the calculated objective in non-sensory areas and the predefined minimum value. Responsive to determining, at step 250, that the calculated objective in the non-sensory areas has not reached the predefined minimum value, then it may be concluded that the optimization process has not yet achieved the desired level of neural activity in these regions. In such a situation, an embodiment may, at step 255, no longer consider the non-sensory objective for further analysis in the current optimization iteration. The optimization process continues using only the sensory objective for the next iteration. This decision acknowledges that the non-sensory areas might not yet exhibit the desired neural responses. Conversely, responsive to determining, at step 250, that the calculated objective in the non-sensory areas has reached the predefined minimum value, that the calculated objective in the non-sensory areas has reached or exceeded the predefined minimum value, then it may be concluded that the optimization process has achieved a satisfactory level of neural activity in these regions. In such a situation, an embodiment may, at step 260, utilize the amount over the minimum (i.e., the surplus achieved beyond the predefined minimum value) in the non-sensory objective and average it with the sensory objective calculated in the same iteration. The averaged objectives serve as guidance for adjusting the stimuli parameters in the next iteration of the optimization process. The goal may be to refine the stimuli to further enhance the overall neural activity in both the sensory and/or non-sensory areas. More particularly, the optimization process gains the advantage of considering both the sensory and non-sensory contributions, which may potentially lead to more refined and effective adjustments in stimuli parameters over successive iterations.

At step 265, an optimal stimulus may be selected and the optimization process may conclude. In an aspect, the optimization process may involve multiple iterations where stimuli parameters are adjusted based on calculated objectives related to neural responses in both sensory and non-sensory areas. The specific number of iterations may be defined manually or may be determined dynamically, e.g., by the system. Throughout this closed-loop optimization process, the progress of the calculated objectives is continuously monitored. In an aspect, the optimization process may include criteria to check for objective stagnation. In this regard, the system may be configured to determine if the objectives have plateaued or are no longer showing significant improvement over a specified number of consecutive iterations. If no improvement is detected, then the system may conclude that further adjustments to stimuli parameters are unlikely to yield substantial benefits. Such a determination may trigger the optimization process to stop and subsequently select the stimulus and/or the stimulus parameters that had reached the highest objective throughout the iterations.

This stimulus may be considered optimal as it induced the most favorable neural responses according to the defined objectives.

### Methods for Diagnosing Neurological Disorders with Visual System Function Based Diagnostics

Certain neurological disorders, even those that interact with a sensory system, may present particular diagnostic or therapeutic challenges. Disorders such as visual neglect or autism often result from strokes or developmental factors, and may manifest as complex and heterogeneous conditions that necessitate accurate and nuanced diagnostic approaches. Conventional diagnostic tests for these disorders are rudimentary, providing limited insights into the intricacies of an individual's condition.

For instance, the standard diagnostic tools for visual neglect often include bedside tests such as line bisection, line cancellation, and drawing tasks. While these tests are quick and commonly employed, they are static, rely on heuristically designed stimuli, and primarily depend on behavioral responses. This reliance on behavioral observations may introduce errors and limit the accuracy of diagnosis. Moreover, these conventional methods might not uncover mild or previously undescribed variants of the disorders due to their limited scope and lack of sensitivity. Similar limitations exist in conventional diagnostic approaches in the case of autism and other neurological disorders impacting visual processing.

The shortcomings of conventional diagnostic approaches generally stem from their reliance on static stimuli and behavioral responses. These methods often lack the capability to explore a broad stimulus space, including dynamic stimuli, and to measure neural responses comprehensively. As a result, the diagnostic accuracy is compromised, leading to an incomplete understanding of the unique characteristics of each individual's disorder.

The concepts described herein introduce an improvement over conventional diagnostic for neurological disorders affecting the visual system. By introducing high-fidelity functional mapping through the creation of specialized maps using EEG and/or MEG, the disclosed methods may identify the intricacies of an individual's visual system function. Such maps, which may be relatively high-dimensional (for example, more than 100 dimensions), may be projected onto low-dimensional (for example, fewer than 10 dimensions) metrics, which may thereafter be associated with known diagnoses. The resulting models for classification, clustering, and scoring enhance diagnostic sensitivity, offering a dynamic and comprehensive understanding of visual system-related disorders.

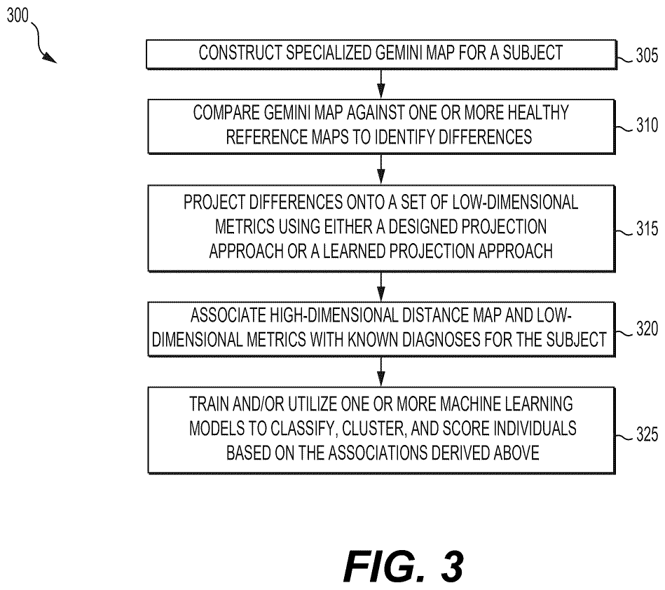

Referring now to FIG. 3, an exemplary workflow 300 is provided for diagnosing neurological disorders with visual system function based diagnostics.

Aspects of the exemplary workflow 300 may be performed in accordance with some or all components described in FIGS. 1A and 1B.

At step 305, a specialized map may be constructed for an individual subject. In an aspect, this construction process may begin through the acquisition of neural data through EEG and/or MEG techniques. These non-invasive techniques capture the electrical and magnetic activity, respectively, occurring in the brain. As previously mentioned above, EEG records electrical activity using electrodes placed on the scalp, while MEG measures magnetic fields generated by neural activity. Each technique may determine neural data generated by the occipital lobe, the region responsible for visual processing. The gathered neural data may then be processed to create a detailed and comprehensive functional map, which may effectively provide a representation of how the individual's visual system encodes dynamic information. Specifically, the constructed map may represent the coordinated dynamic neural responses across combinations of various subregions of the occipital lobe or other regions, highlighting processing circuits of heightened or reduced activity during, as relevant, visual processing. For simplicity purposes, this constructed map is described herein as a “Gemini” map. It is important to note that the Gemini name is for designation purposes only and is not intended to imply any functional limits, or other inferences, associated with this constructed map. In an aspect, the construction of the Gemini map, or “stimulus map,” may be facilitated as described below.

In one aspect, the disclosed embodiments provide a method for providing spatiotemporal sensory inputs to one or more participants to produce a stimulus map of the brain. The method includes sampling a spatiotemporal sensory code generation model with a first encoding vector to produce a first spatiotemporal sensory code in the form of a first video sequence. The method further includes outputting the first video sequence to provide a first spatiotemporal sensory input to said one or more participants. The method further includes receiving one or more neural response measurements for said one or more participants, said one or more neural response measurements being performed while the first spatiotemporal sensory input is being presented to each respective one of said one or more participants. The method further includes determining an outcome function based, at least in part, on said one or more neural response measurements for said one or more participants. The method further includes producing a second encoding vector based, at least in part, on the first encoding vector and the outcome function. The method further includes iteratively repeating said sampling, said outputting, said receiving, and said determining with the second encoding vector, and any successive encoding vectors, until a defined set of stopping criteria for the outcome function is satisfied. Upon satisfying the defined set of stopping criteria for the outcome function, a resulting spatiotemporal sensory code is stored to form part of a stimulus map of the brain.

Embodiments may include one or more of the following features, separately or in any feasible combination.

The spatiotemporal sensory codes may include one or more of the following: visual sensory inputs, auditory sensory inputs, and somatosensory inputs. The generation model may include procedural graphics using input parameters including one or more of: spatial frequencies, temporal frequencies, spatial locations, spatial extents, and translation-based motion vectors. The spatiotemporal sensory code generation model may include a generative adversarial network or deep diffusion model and the first encoding vector may point to a location in a latent generation space.

The spatiotemporal sensory codes, in the form of video sequences, have a defined time length and partially overlap in time. The first video sequence may have N frames starting from time Ti, and the method may further include: applying a per-frame window function to the first video sequence; and adding the result to an output frame buffer, filling frames from Ti to Ti +N. The successive encoding vectors may be produced based at least in part on the outcome function and a plurality of preceding encoding vectors.

The producing of the second encoding vector may be done at time Tᵢ+S, where S<=N, and the method may further include: applying the per-frame window function to the second video sequence; and adding the result to the output frame buffer, resulting in the output frame buffer comprising frames Tᵢ to Tᵢ+S+N. During said outputting, frames from Tᵢ to Tᵢ+S may be output from the output frame buffer to be presented to said one or more participants while the second video sequence is being produced.

The outputting may include displaying said sequence of spatiotemporal sensory inputs to one or more electronic screens. The one or more neural response measurements may be performed using one or more of the following: EEG, quantitative EEG, MEG, single-photon emission computed tomography (SPECT), positron emission tomography (PET), functional magnetic resonance imaging (fMRI), and functional near-infrared spectroscopy (fNIRS). The one or more neural response measurements may be received from a multiple-channel buffer including current multiple-channel neural response measurements and previous multiple-channel neural response measurements

The method may further include aligning timewise, across said one or more participants, said one or more neural response measurements; extracting one or more features for each measurement time step across said one or more neural response measurements and across said one or more participants; and comparing said one or more extracted features to targets to calculate the outcome function. The defined set of stopping criteria may include one or more of the following: specified convergence criteria, a specified number of iterations, and a specified amount of time.

In storing said resulting spatiotemporal sensory code to form part of the stimulus map of the brain, a feature representation of said one or more neural response measurements may be associated with a location in a high dimensional space. The resulting spatiotemporal sensory code may be associated with a neural state at a specific brain location. The resulting spatiotemporal sensory code may be associated with a whole-brain neural state. The whole-brain neural state may be defined in terms of multivariate cross-coherence across spectral bands and said resulting spatiotemporal sensory code may be adapted to maximize the cross-coherence across one or more pairs of nodes of the brain map.

In another aspect, the disclosed embodiments provide a system for providing spatiotemporal sensory inputs to one or more participants to produce a stimulus map of the brain. The system includes at least one processor; and at least one non-transitory processor-readable medium that stores processor-executable instructions which, when executed by said at least one processor, cause the at least one processor to perform the methods discussed above.

At step 310, the Gemini map may be compared against one or more reference maps derived from healthy individuals, which may represent typical neural activity within the visual systems during visual processing. The maps may also include those derived from individuals with known medical conditions. In an aspect, this comparison may be conducted in a multidimensional map space, considering various dimensions that capture the complexity of neural responses in the occipital lobe. This multidimensional approach allows for a nuanced examination of differences, as opposed to a simplistic comparison based on a single metric. In an aspect, a difference operation method may be employed in which the Gemini map constructed in step 305 may be subtracted from the composited healthy maps, and the magnitude of the difference may be subjected to a threshold which may be dependent on the variance of the random spontaneous activity at that map location and/or the variance of the responses to identical stimulus conditions. This operation may result in a map that highlights the magnitude and sign of the specific differences (e.g., areas of divergence or convergence) between the individual's neural activity and the healthy composited maps.

At step 315, the maps or the observed differences may be projected onto a set of low-dimensional metrics. In an aspect, these metrics may serve as a condensed representation of the various aspects of divergence or convergence in neural activity and are designed to summarize specific features related to visual system function, thereby providing a more interpretable and manageable set of parameters for further analysis. Step 315 describes two exemplary methods of how this projection process may be facilitated.

A first exemplary method of projecting the differences or similarities, e.g., a “Designed Projection,” may be achieved via expert input. Specifically, by capturing key aspects of neural processing, such as processing speed and data throughput, a more focused analysis of the subject's visual system abnormalities may be achieved.

In a first sub-step, temporal aspects of neural processing may be captured, particularly those related to the speed of information processing within the visual system. Specific portions of the Gemini map may be selected by an expert that reflect response latency and/or oscillatory frequency. By applying a first weighted average to these selected portions, the resulting metric represents the speed at which the subject's visual system processes information.

In a second sub-step, the efficiency and information flow within the visual system may be assessed. In this regard, a second weighted average metric may be designed by an expert by evaluating portions of the Gemini map that reflect the power of cross-spectrum across known processing hierarchies, including visual system and cognitive processing. This metric provides insights into the data throughput, or the capacity of the visual system to transmit and process information.

A second exemplary method of projecting the maps or differences, e.g., a “Learned Projection,” represents a data-driven approach where the projection is learned based on a different strategy to rate low-dimensional metrics for subjects. This method may be especially useful when there is a sufficiently large database of subjects (e.g., over 100 subjects) available. In an aspect, subjects may undergo behavioral tests that rate their speed of visual processing. The goal is to optimize a map projection across these subjects to predict speed of processing scores. This optimization process involves adjusting the map projection such that the projected maps or projected differences observed between the Gemini map and the healthy maps align with the behavioral metrics. In an aspect, constrained optimization techniques may be applied to ensure the projection adheres to specific criteria.

Additionally or alternatively to the foregoing, the Gemini map may be provided into a machine learning model, e.g., such as a random forest or a Multi-Layer Perception (MLP). The model may be trained on features of the Gemini map or map differences to learn the relationship between the features of the constructed map and the low-dimensional metrics. This trained model may then be used to predict scores for new individuals based on their own constructed maps.

At step 320, the high-dimensional maps and the low-dimensional metrics may be associated with known diagnoses of the subject. These associations may not only aid in confirming or refining existing diagnoses, but may also serve as a foundation for extending the diagnostic capability to new individuals with potentially different or lesser-known conditions. In an aspect, the high-dimensional distance map, created by comparing the subject's constructed Gemini map with healthy composited maps as described above in step 310, provides a spatial representation of the deviations or similarities in neural activity. This map, coupled with the low-dimensional metrics derived in step 315, serves as a comprehensive characterization of the subject's visual system function.

At step 325, one or more machine learning models may then be trained, e.g., using one or more supervised learning approaches, to classify, cluster, and score new individuals based on the associations made with a dataset of individuals with known diagnoses, as further described below.

In an aspect, a dataset containing information on individuals with known diagnoses may be utilized for training a classification model. The dataset may include individuals with a range of neurological disorders affecting the visual system, each associated with their high-dimensional distance maps and low-dimensional metrics. In an aspect, if both the constructed Gemini map and the low-dimensional metrics are utilized in the classification process, feature extraction may be performed. In this regard, the Gemini map may be downsampled to have no more than, for example, 10,000 data points, and the map may be averaged over each axis (e.g., for a 3D map, averaging the map over x, y, and z axes). In an aspect, classification models, such as Random Forest models, may be employed for this task. These models are appropriate for scenarios with lower numbers of training samples (e.g., 100 to 1000), making them suitable for neurological disorders that may have limited available data. In an aspect, the classification model may be trained on the dataset, learning to recognize patterns and associations between the input features and the known diagnoses. The model may then be validated using a separate set of data to ensure its generalizability.

In an aspect, once the model is trained and validated, it may be applied to new individuals. The Gemini map and the low-dimensional metrics of the respective individual may be input into the model, and the model may predict the most likely diagnostic category based on the learned patterns from the training dataset. The output of the classification process may provide a diagnostic category for the individual, indicating the likely neurological disorder affecting their visual system. This information may aid clinicians and healthcare professionals in making informed decisions regarding treatment plans, interventions, and ongoing care.

In an aspect, individuals who share similar score profiles may be clustered together into groups. Clustering helps to identify patterns and similarities in visual system function that may not align with predefined diagnostic categories, potentially revealing novel or undefined diagnoses. Standard clustering algorithms, such as K-means clustering, may be employed for this purpose.

In an aspect, individuals may be scored on the severity of the diagnosed neurological disorder. Similar to the classification and clustering steps described above, features may be extracted from the constructed map and the low-dimensional metrics. Downsampled maps and averaged metrics over specific axes may be utilized as features. Unlike classification, which assigns individuals to discrete categories, regression models may be employed for scoring. For instance, regression models, such as linear regression or other regression algorithms adapted for the task, may be trained to predict a continuous severity score based on the extracted features. Once trained, the regression model may be applied to new individuals. The features extracted from the individuals'constructed map and the metrics of their personalized low-dimensional metrics may be input to the model and the model may predict their severity score. This score quantifies the severity of the diagnosed neurological disorder for that individual.

### Use of Spontaneous Brain Activity to Constrain Sensory Inputs Used to Control Brain Activity

EEG and MEG are technologies that may provide valuable insights into the dynamic interplay of neural processes within the brain—offering a window into the complex world of cognitive functions and sensory perception. However, a significant challenge persists in identifying and manipulating the specific sensory inputs that can effectively control brain network activity. In nonlinear systems and networks, the search for such inputs becomes particularly daunting, often requiring exhaustive exploration of a vast space of possible inputs.

Conventional approaches to this problem involve conducting experiments with human participants, exposing them to a range of sensory stimuli, and monitoring resultant brain activity using EEG or MEG. This process, while informative, is resource-intensive and lacks efficiency. Additionally, in linear systems, superposition properties allow for combining responses to individual stimuli to predict the response to a combination of stimuli. However, these properties may be weak or absent in nonlinear brain networks, complicating the identification of effective stimuli combinations. Furthermore, conventional attempts at finding dynamic similarity between brain activity and sensory inputs often rely on simplistic metrics, such as autocovariance matrices. These methods may overlook nuanced patterns and relationships in the data.

Techniques described herein reduce search time and enhance the organization of the search space. Firstly, the use of dynamic similarity measures, such as advanced statistical methods and predictor-based approaches, provides a more nuanced understanding of the relationship between sensory inputs and brain activity. This leads to a more targeted and efficient search process. Secondly, the described concepts focus on organizing the search space to increase smoothness, acknowledging that adjacent points in the space are more likely to exhibit similar control properties. By learning projections that place similar dynamics closer together and leveraging transformations of brain activity, certain aspects introduce a structured and systematic way to navigate the input space.

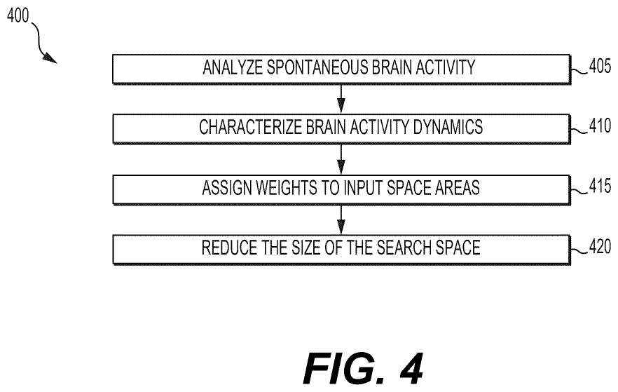

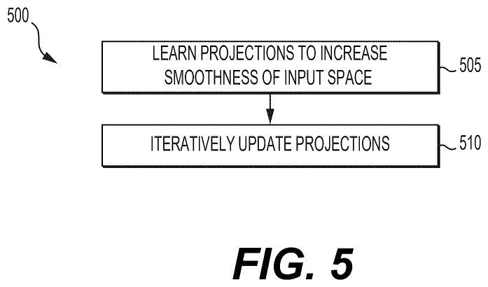

Referring now collectively to FIGS. 4 and 5, exemplary workflows 400 and 500 are provided for utilizing spontaneous brain activity to constrain sensory inputs used to control brain activity. In particular, exemplary workflows 400 and 500 may be performed in accordance with some or all components described in FIGS. 1A and 1B.

Relevant terminology related to this section may include the terms described below.

A “large space of possible inputs” may correspond to one sensory input may be uniquely characterized by a list of unique values characterizing the input or used to create the input. This represents one point in a space of possible inputs. The list of unique values can be parameters that are used in an algorithm utilizing parametric functions (such as Gaussian functions or Gabor functions). One input may have dynamical properties, such that it spans a period of time, such as a video clip, an audio clip, or a somatosensory spatio-temporal stimulus clip.

“Brain activity” may be multi-electrode/channel EEG or MEG (or other brain recording modalities that have a temporal component that conveys significant information about neural activity). The activity may have a temporal component, generally a time series regularly sampled at a fixed rate, for example 1kHz. The activity may have a spatial component, either in “signal space” where electrodes represent points in space, or in “source space” where a source localization model such as low-resolution electromagnetic tomographic analysis (LORETA) may be applied which maps the collection of channel time-series into “voxel” time series where each voxel is a 3D spatial point in the brain. The spatio-temporal activity can be mapped onto basis functions that are either designed or learned, representing a library of potentially complex spatio-temporal component patterns. These can be learned with Principal Component Analysis (PCA), independent component analysis (ICA), or deep representation learning, or they can be wavelet or sinusoidal Fourier components (for example). The output of the basis function projection may be real valued or complex valued. Activity may be a function of multiple channel activity over some time period, for example a graph describing pairwise cross correlations between channels in a variety of basis-function bands. Subsets of the graph in subsets of basis functions can be thought of as independent channels of activity. Such channels may be defined or learned, for example to minimize similarity between channels under certain constraints, such as similarity is only measured when other conditions are met, such as a particular EEG microstate is active or other independently defined states based on tasks, behavioral observations, or brain activity metrics. An instance of brain activity may be defined with respect to a time period. This time period might not be fixed. Two time periods may have different durations, each resulting in an independent observation of brain activity. The time periods may be based on when certain conditions are met, such as a particular EEG microstate is active or other independently defined states based on tasks, behavioral observations, or brain activity metrics. The methods here can apply to such non-fixed time durations.

“Control of brain activity” may correspond to properties of brain activity that can be explained to be a function of the sensory inputs. These can be characterized by a vector.

“Similar properties of control” may correspond to if the control of brain activity may be treated as an N-dimensional vector, two sensory inputs have similar properties of control if the distance between the two vectors are closer than their distance to the vast majority of other sensory inputs. Or, if the proximity between the vectors is small compared to the vector space spanning all of the sensory inputs.

“Spontaneous brain activity” may correspond to brain activity recordings of unconstrained viewing/listening/behaving. May be obtained from repositories containing such recordings from numerous healthy people. Should not be obtained from specialized, difficult, or focused activities, or from people with brain disorders.

In an aspect, two independent processes will be described herein that may enable the search of relevant input stimulus to be more achievable.

A first process may relate to reducing the average time needed to search for sensory inputs that control brain activity by more highly weighting areas of the input space much more likely to yield results. In cases where some of the weights are zero, the size of the search space may be strictly reduced, otherwise it is effectively reduced. This may be done in various ways. For instance, in an aspect, weights may be derived by analyzing spontaneous brain activity and assigning higher weights to parts of the input space that have dynamics that are more similar to the dynamics of the brain activity.

A statistics-based method is described herein for identifying the dynamic similarity between a given input space and brain activity. Dynamics (e.g., time-series statistics) may be characterized in various known ways, and similarity between dynamical or stochastic processes may be characterized in various known ways. One simple way may be an autocovariance matrix of the “brain activity” (see definition-can be signal space, source space, and after a basis-function projection), which may be matched to the autocovariance matrix of each point in the input space (similarly, the input autocovariance may be multi-channel where a channel is a spatial location and/or various basis function activations after a projection, including random permutation projections) using a Euclidean distance metric. Another technique may compare Eigenvalue decompositions. Additionally or alternatively, methods for comparing the higher-order statistical properties of nonlinear dynamical properties may be more effective in some situations. Examples of this include comparisons between Markov Chain models of each process, or Information Theoretic based comparisons.

A predictor-based method is also described herein for identifying dynamic similarity between a given input space and brain activity. In this method, weights may be the degree to which one process or the statistics of one process may be predicted from the other process, under certain constraints of the prediction architecture. This may be done, for example, by training a small recurrent neural network (RNN) (one of many example model architectures which can predict and generate time-series) to predict the process of the input-space, and to measure the degree of prediction of the same RNN to predict the spontaneous brain activity while only retraining the output fully connected layer for a small, fixed number of learning iterations.

In an aspect, an input space may be learned with joint objectives of dynamic similarity to brain activity and desired sensory input statistics. This concept can be generalized to the case where the input space is learned, which is an effectively reduced space for controlling brain activity. In this approach, the RNN (again, one of many example model architectures which can generate time-series) may start with random weights, and these weights may be iteratively adjusted (for example using stochastic gradient descent (SGD)) to maximize the dynamics similarity between the generated process and the spontaneous brain activity. Starting repeatedly from various random RNN initializations can ensure a breadth of the input space, and each point of the input space can be characterized by either the converged or initialized RNN coefficient values or some function of them (for example a low dimensional projection via principal component analysis (PCA)). The generator may involve a projection through some learned or designed basis functions. These may be learned, for example, to produce inputs that match a desired statistical property of the inputs, such as spatial frequency distributions, spatial cross-covariances, activation of a computational model of visual cortex, and the like, where “spatial” (which may apply to visual, auditory/tonotopic, and somatosensory space) may be extended to spatial and other projections (spatial-spatial frequency, spatial-spatial basis function/wavelet, etc.).

In an aspect, an input space may be learned based on dynamics-preserving transformations of brain activity that optimize desired sensory input statistics. The input space may be learned by learning transformations of the brain activity. In this method, spontaneous brain activity—or outputs of a model trained to generate spontaneous brain activity—may be passed through a random static (operations are only applied on the non-temporal axes) transformation which may be linear (e.g., random permutation matrix) or nonlinear (randomly initialized deep neural network) that maps the brain activity channel/basis function domain to the input channel (e.g. space)/basis function domain. Basis functions on the input domain projection may be designed or learned to match a desired statistical property of the inputs, such as spatial frequency distributions, spatial cross-covariances, activation of a computational model of visual cortex, and the like, where “spatial” (which may apply to visual, auditory/tonotopic, and somatosensory space) can be extended to spatial and other projections (spatial-spatial frequency, spatial-spatial basis function/wavelet, etc.). The input space points may be characterized by the coefficients of the random projection or the activations of the designed or learned basis functions, or some function of them (for example a low dimensional projection via PCA).

In an aspect, weights may be updated based on control experiments.

Such sensory input space weightings based on spontaneous brain activity may be updated according to the observed degree of control of brain activity established between that point in the input space and the brain activity. Ultimately, the weighting may be learned based on the multiple observations of degree of control and input features describing the input space and the spontaneous brain activity, for example using a ML model trained to perform regression using the observed weights as targets.

In an exemplary process flow related to the foregoing, at step 405, spontaneous brain activity may be analyzed. In an aspect, data may be utilized from spontaneous brain activity, e.g., recorded through EEG or MEG, obtained from one or more data locations. The brain activity data may be cleaned and preprocessed to isolate relevant patterns and features.

At step 410, the brain activity dynamics may be characterized. In an aspect, various time-series statistical methods may be employed to characterize the dynamics of spontaneous brain activity. This may include measures such as autocovariance matrices, Eigenvalue decompositions, and/or higher-order statistical properties.

At step 415, weights may be assigned to input space areas. In an aspect, for dynamic similarity weighting, weights may be derived by analyzing spontaneous brain activity and assigning higher weights to areas in the input space with dynamics more similar to those of the brain activity. In another aspect, for Euclidean distance metric weighting, a Euclidean distance metric may be utilized to match the autocovariance matrix of brain activity to each point in the input space, emphasizing areas with closer dynamic resemblance.

At step 420, the search space may be reduced. More particularly, depending on whether some weights are zero or non-zero, the size of the search space may either be strictly reduced (if some weights are zero) or effectively reduced (if all weights are non-zero). This reduction streamlines the search process.

A second process may relate to organizing the search space to have increased smoothness. In the context of this application, “smoothness” may indicate that the value of adjacent points tend to be close together, e.g., the local derivative across the direction between the points is small, or within a predetermined threshold. Additionally, “value” may refer to some measurement of the resulting response, such as amplitude, frequency, and/or phase More particularly, in an aspect, a smooth space may indicate that adjacent points in the space are more likely to have similar properties of control, and when one moves in a direction between two adjacent points in the space, the change in control properties are more likely to be predictive of the change between the second point and a third point being the next step in the same direction. This may be completed in various ways, including creating smooth input spaces or increasing the smoothness of input spaces for applications described herein.

In an aspect, a projection may be learned that places input space points closer to one another when their dynamics have greater similarity (for example using the dynamics similarity measures described herein). The projection may preserve the original space dimensionality, or it may reduce it. In an aspect, this method may be extended by iteratively updating the projection based on observations of the pairwise distance between the corresponding brain control properties. The warping itself may have smoothness constraints that attempt to preserve distances in the original projection, i.e. control properties would have to be repeatedly observed to be very different with high certainty in order for originally very close points to move far apart.

In an exemplary process flow related to the foregoing, at step 505, projections may be learned to increase smoothness of the input space. In an aspect, a projection method may be defined that organizes the input space points in a way that those with greater dynamic similarity (e.g., similar brain activity control properties) are brought closer together. In an aspect, it may be determined whether the projection preserves the original space dimensionality or reduces it. This decision may depend on specific characteristics of the input space and the desired outcomes.

At step 510, the projections may be iteratively updated. In an aspect, observations may be collected of the pairwise distance between corresponding brain control properties for different input space points. The projections may be iteratively updated based on these observations to ensure that similar brain control properties remain close in the projected space. In an aspect, certain smoothness constraints may be implemented to minimize distortions in the input space.

### Neural Signal-to-Noise Ratio (SNR) Maximization for Rapid Closed-Loop Optimization Using Short Time Analysis Windows and Hyperscanning

The reliable interpretation of neural signals associated with brain functionality is important for understanding cognitive processes, identifying biomarkers, and developing effective interventions for neurological disorders. However, the accurate extraction of neural information faces significant challenges due to the presence of noise and the limitations of conventional observation techniques. Traditional methods such as EEG and MEG are fundamental tools for studying neural activity. However, these methods often produce observations containing both signal and noise, making it challenging to discern the relevant neural information. The SNR serves as a critical metric, quantifying the extent to which the signal of interest is overshadowed by unwanted noise. In the pursuit of meaningful insights, one technique resorts to employing long time windows and multiple trials, leading to prolonged experimental durations and potential oversights in capturing dynamic natural events.

To achieve large enough SNR to infer functionality, techniques may either use “biomarkers,” which are defined over long time windows (such as “alpha frequency strength”), or average the activity across repeated trials of the same reference state. Reference states may be induced by a particular sensory input, or motor task, or cognitive task. Often, both long analysis time windows and multi-trial averaging are used together to find some biomarker with sufficient SNR which is hoped to infer functionality or changes in functionality.

However, such practices may limit the extent of functionality that can be observed. In naturalistic continually evolving behavioral settings, the brain is constantly and rapidly adjusting neural assemblies that dynamically connect different neural populations across the brain. Observing the functionality of such network behavior requires short time windows. In such naturalistic settings, there also may be much more information content in the fine-time structure in neural signals compared to that contained in singular oscillation frequencies or overall activity magnitude observed across long time windows. Another disadvantage of multiple-trial averaging is that the strongest and most detailed aspects of neural signal are often present in the first presentation of a sensory input, while subsequent presentations of the same input lead to weakened or absent neural activity.

In combination, using long time windows and multi-trial averaging slows scientific progress to impractical levels. If biomarkers require seconds-long activations to be collected, and averaging across dozens of trials, the amount of time required is 2-3 orders of magnitude slower than if activity patterns could be observed over tens to hundreds of millisecond time windows in continuously evolving (non-repeating) inputs. For applications of closed-loop optimized neurofeedback, such practices may prevent convergence or at least greatly slow convergence to a suboptimal state. If such neurofeedback involves achieving an effect that generalizes across people, the situation becomes another order of magnitude more laborious. In effect, the large majority of possible benefits of closed loop neurofeedback systems cannot be practically achieved.

The concepts described herein combine hyperscanning, short time windows, and/or an alignment function, to provide a comprehensive solution to maximize SNR in neural activity, enabling more accurate and rapid closed-loop optimization in neuroscience research and applications. In the context of this application, hyperscanning may refer to neuroimaging multiple people substantially simultaneously, e.g., in response to the same synchronized stimulus. More particularly, hyperscanning techniques may be incorporated to integrate neural signals from multiple subjects concurrently exposed to identical stimuli. This technique may utilize short time windows (e.g., 10-100 ms) for observing neural events, thereby capturing rapid adjustments in neural assemblies. Additionally, a spatial alignment function may be utilized that optimizes weights for each channel, ensuring optimal integration of neural signals across subjects. The alignment function effectively institutes temporal alignment using per-channel delays or dynamic time-warping, compensating for individual differences in brain structure and electrode positioning.

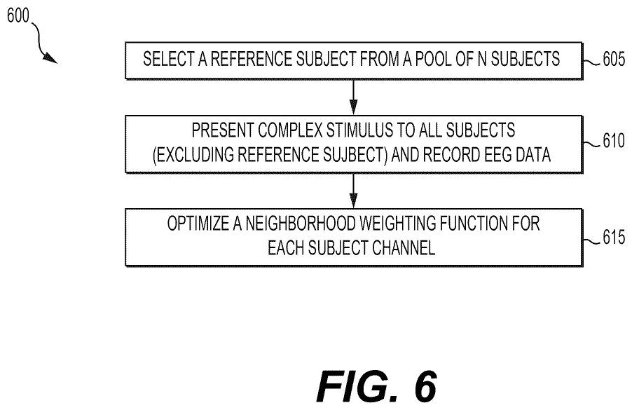

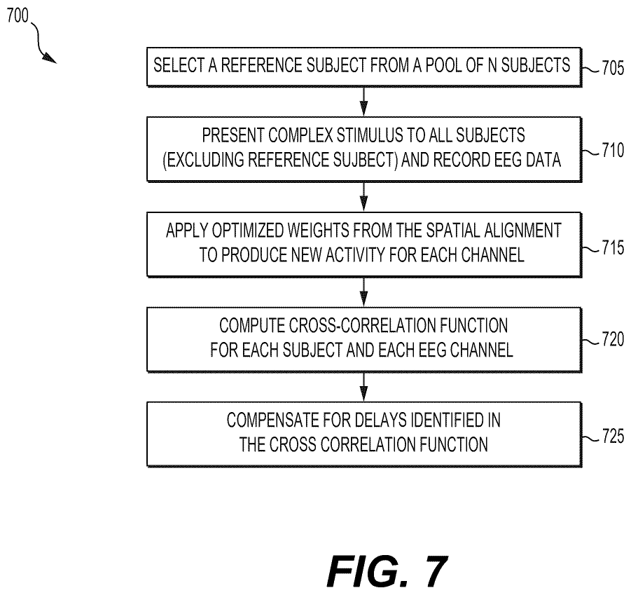

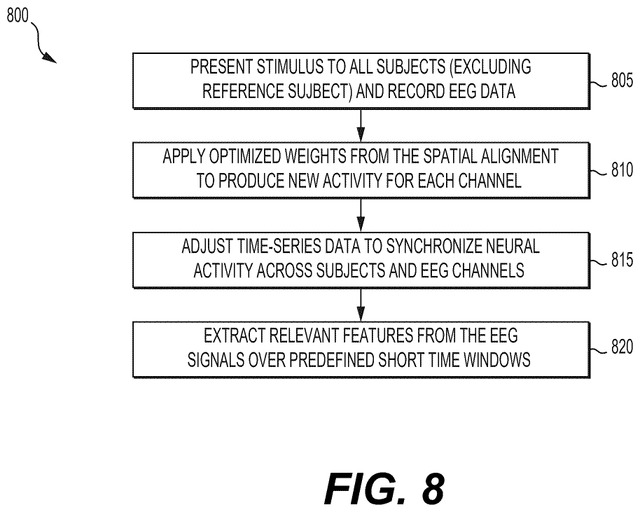

Referring now collectively to FIGS. 6-8, exemplary workflows 600, 700, and 800 are provided for utilizing short time analysis windows and hyperscanning for neural SNR maximization for closed-loop optimization. Aspects of the exemplary workflows 600, 700, and 800 may be performed in accordance with some or all components described in FIGS. 1A and 1B.

Provided below are a series of steps that may be followed to design a spatial alignment function.

At step 605, one or more reference subjects from a pool of N subjects may be selected to serve as a baseline against which the neural signals of other subjects may be compared and aligned. In an aspect, the reference subject may be representative of an individual whose neural signals will be used as a standard for alignment. In an aspect, consideration may be given to factors such as age, gender, health or disease status, anatomical similarity, and/or overall neurological characteristics to minimize bias in the alignment process. In general, an ideal reference subject may be one whose neural responses are expected to be consistent and representative of a specific group, thereby enhancing the effectiveness of the alignment process.

At step 610, a complex stimulus may be presented to all subjects (except for the reference subject) and their EEG's may be recorded. In an aspect, a “complex stimulus” may refer to a sensory input that is rich and engaging, often used to elicit diverse neural responses. This may include natural videos, audiovisual sequences, or other stimuli tailored to the research objectives. In an aspect, the choice of stimulus may be dictated by the specific goals of the experiment (e.g., to study cognitive processes, sensory perception, motor tasks, etc.). In an aspect, stimuli may be presented simultaneously to all subjects to create a synchronized experimental environment. This may ensure that all participants are exposed to the same sensory input at the same time. In an aspect, the experimental conditions, e.g., lighting, sound, any other relevant environmental factors, may be controlled to minimize extraneous variables that may impact the neural responses. In an aspect, EEG recording equipment may be employed to capture the electrical activity of the brain during the presentation of the stimulus. Once setup is complete and the relevant parameters have been established (e.g., sampling rate, filter settings, etc.), EEG data may be recorded. In an aspect, EEG data may be recorded for the entire duration of the stimulus presentation. Alternatively, in another aspect, the recording may occur over a shorter duration, e.g., based on the design of the experiment.

At step 615, a neighborhood weighting function may be optimized for each channel, thereby ensuring that the cross-correlation between an individual subject's channel and the corresponding channel of the reference subject is maximized. In an aspect, the neighborhood weighting function may assign weights to each neighboring channel of a given channel. These weights determine the contribution of each neighboring channel to the cross-correlation with the corresponding channel of the reference subject. In an aspect, a goal may be to maximize the height of the cross-correlation peak between a channel and the reference subject's corresponding channel. The weights may be adjusted during an optimization process to enhance the alignments accuracy. In an aspect, neighboring channels may include immediate neighbors, second nearest neighbors, or a broader set of channels depending on the chosen spatial context. In an aspect, to ensure that the weights contribute proportionally to the overall response, they may be normalized so that their sum equals 1. This normalization may prevent one channel from dominating the aligned channel. In an aspect, the optimization process may involve constraints to ensure that the weights adhere to specific criteria. For example, constraints may be applied to prevent negative weights or to limit the maximum weight assigned to a neighboring channel.

Two approaches are described below for designing a temporal alignment function. Approach one may be directed to a “per-channel delay” technique whereas approach two may be directed to a “dynamic time warping” technique.

With respect to the first approach, at step 705, one or more reference subjects from a pool of N subjects may be selected to serve as a baseline against which the neural signals of other subjects may be compared and aligned. Step 705 may be similar to step 605, the details of which are described above.

At step 710, a complex stimulus may be presented to all subjects and their EEG's may be recorded. Step 710 may be similar to step 610, the details of which are described above. Additionally or alternatively, the chosen complex stimuli may be different to be more optimal for the spatial alignment or the temporal alignment and/or the stimulus duration may be different between the two steps.

At step 715, the optimized weights (e.g., obtained from the spatial alignment determined at step 615) may be applied to produce new activity for each channel.

At step 720, the cross-correlation function may be computed for each subject and each EEG channel. In an aspect, the cross-correlation function quantifies the similarity between two signals as a function of the time lag between them. In this context, it measures the similarity between the EEG signals of a subject (except the reference) and the corresponding channel of the reference subject. The computation may be performed separately for each EEG channel, comparing the signal from the channel of interest in the subject to the corresponding channel in the reference subject.

In an aspect, the time lag of the peak of the cross-correlation function indicates the temporal misalignment or delay between the signals. A positive time lag signifies that the signal in the subject's channel is delayed concerning the reference subject's channel, whereas a negative time lag indicates an advancement. In an aspect, a threshold (e.g., such as 0.4, for peak values ranging from 0 to 1) may be applied to determine whether the identified peak is significant. Peaks below the threshold may be considered negligible or indicative of noise, and their corresponding delays may be marked as “missing.” In an aspect, the shape of the cross-correlation function may provide additional insights into the temporal relationship between signals. For example, broader peaks may indicate more gradual changes in alignment. Statistical analysis may be applied to assess the significance of identified delays or to compare delays distributions across subjects.

At step 725, delays identified in the cross-correlation function may be compensated for, particularly for channels where the peak is above the threshold. In an aspect, for each subject (except the reference subject), the delays identified for each EEG channel are compensated for, aligning the signals with the corresponding channels of the reference subject. Compensation may be achieved by applying a delay filter or shifting the samples of the EEG signal by the appropriate number of samples corresponding to the identified delay. In an aspect, various methods may be employed for delay compensation, including finite impulse response (FIR) filters, infinite impulse response (IIR) filters, or other signal processing techniques. For channels where the peak of the cross-correlation function did not surpass the threshold (i.e., “missing” delays), interpolation methods are employed to estimate these delays. For example, linear interpolation, spline interpolation, or other mathematical techniques may be utilized to predict missing delay values. In certain cases, extrapolation may be used to estimate delays for channels with missing information, extending the compensation process beyond the observed data. In an aspect, certain characteristics of the EEG signal, such as its frequency content, amplitude variations, and SNR, may influence the choice of interpolation or extrapolation.

With respect to the second approach, DTW is a powerful technique used for temporal alignment in the context of hyperscanning. This approach is particularly valuable when dealing with temporal variations in signals that may not be perfectly aligned due to differences in subjects'responsive times or signal distortions.

In the context of DTW, and at step 805, a stimulus may be chosen that is played simultaneously (e.g., for 10 seconds, etc.) for all subjects and the electrical activity of all members in the subject pool may be recorded by an EEG.

At step 810, the optimized weights (e.g., obtained from the spatial alignment determined at step 615) may be applied to produce new activity for each channel.

At step 815, the time series data may be adjusted to synchronize neural activity across subjects and EEG channels. In an aspect, temporal alignment may typically be performed after spatial alignment, ensuring that EEG signals from different subjects and channels are synchronized in time. The spatial alignment using DTW implicitly facilitates the temporal alignment, as the warping path adapts to align temporal dynamics. In the DTW approach, per-channel delays are determined for each EEG channel of a subject concerning the reference subject. These delays represent the temporal misalignments between channels, accounting for individual differences in response times or temporal variations. If using per-channel delays, delay compensation may be performed with respect to the reference subject using a delay filter, or by shifting samples the appropriate number of samples corresponding to the delay. If using DTW, the DTW algorithm may be applied in each channel with respect to the reference subject.

At step 820, relevant features from the EEG signals may be extracted and/or calculated over short time windows. This process enables the analysis of neural activity patterns in response to the stimulus. In an aspect, the length of the short time window may depend on the experimental goals that are desired to be achieved. For instance, the short time window may range from 10 to 100 milliseconds. In an aspect one or more relevant feature extraction methods may be chosen to characterize neural activity within each short time window. The chosen feature extraction methods may be applied to calculate the relevant features for each short time window within the aligned EEG signals. For instance, an event-related potential (ERP), which corresponds to the measured brain response that is the direct result of a specific sensory, cognitive, or motor event, may be measured by the EEG. A short time-window feature may be one that describes the ERP pattern. This application may result in a set of feature vectors that capture the temporal evolution of neural activity. In an aspect, the calculated features may be averaged across the subjects to obtain group-level information. Alternatively, trends or coincidences may be explored across subjects in the calculated features. This analysis may involve identifying consistent patterns or variations in neural activity across the group.

The functional modules described herein are provided by way of example. It will be understood that different functional modules may be combined to create different utilities. It will also be understood that additional functional modules or sub-modules may be created to implement a certain utility.

In general, any process discussed in this disclosure that is understood to be computer-implementable may be performed by one or more processors of a computer system, such as system environment 110, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 110, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

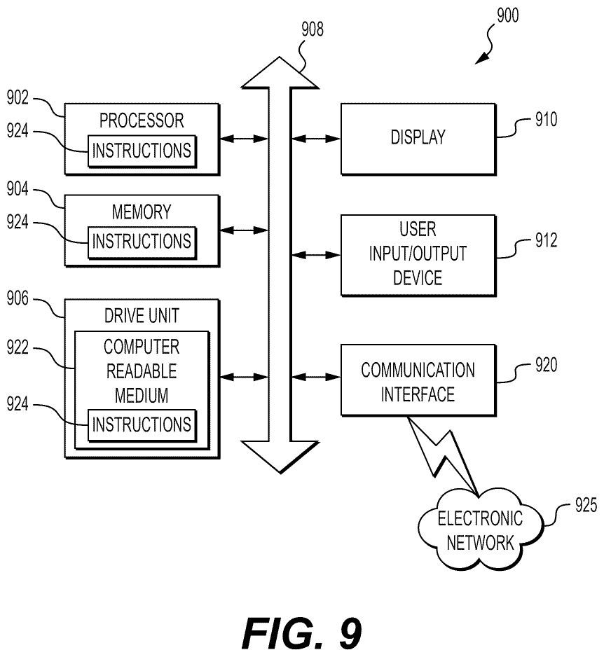

FIG. 9 is a simplified functional block diagram of a computer system 900 that may be configured as a computing device for executing the processes described herein, according to exemplary embodiments of the present disclosure. FIG. 9 is a simplified functional block diagram of a computer that may be configured according to exemplary embodiments of the present disclosure. In various embodiments, any of the systems herein may be an assembly of hardware including, for example, a data communication interface 920 for packet data communication. The platform also may include a central processing unit (“CPU”) 902, in the form of one or more processors, for executing program instructions. The platform may include an internal communication bus 908, and a storage unit 906 (such as ROM, HDD, SDD, etc.) that may store data on a computer readable medium 922, although the system 900 may receive programming and data via network communications via electronic network 925 (e.g., voice, video, audio, images, or any other data over the electronic network 925). The system 900 may also have a memory 904 (such as RAM) storing instructions 924 for executing techniques presented herein, although the instructions 924 may be stored temporarily or permanently within other modules of system 800 (e.g., processor 902 and/or computer readable medium 922). The system 900 also may include input and output ports 912 and/or a display 910 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for performing iterative adjustments to sensory stimuli in a closed-loop optimization system, the computer-implemented method comprising:
receiving, at a computing device, brain activity data associated with a stimulus presented to a subject;
preprocessing, using a processor associated with the computing device, the brain activity data;
performing, using the processor, a source localization technique on the preprocessed brain activity data to identify one or more voxel locations where electrical activity contained in the brain activity data is projected to originate;
grouping, subsequent to performance of the source localization technique and based on the preprocessed brain activity data, a first subset of the one or more voxel locations together, wherein the first subset includes is associated with sensory areas of interest;
grouping, subsequent to performance of the source localization technique and based on the preprocessed brain activity data, a second subset of the one or more voxel locations together, wherein the second subset is associated with non-sensory areas of interest;
determining, using the processor, a first objective function based on localized neural activity identified within the first subset of the one or more voxel locations and determining a second objective function based on the localized neural activity identified within the second subset of the one or more voxel locations;
determining, using the processor, whether the second objective function is equivalent to or exceeds a threshold minimum value;
averaging, responsive to determining that the second objective function is equivalent to or exceeds the threshold minimum value, an amount of the second objective function over the threshold minimum value with the first objective function to produce an averaged objective function; and
utilizing the averaged objective function to refine one or more parameters of the stimulus in a subsequent iteration of the closed-loop optimization system.

2. The computer-implemented method of claim 1, wherein the stimulus is a visual stimulus.

3. The computer-implemented method of claim 1, wherein the one or more parameters of the stimulus include one or more of: a color associated with the stimulus, a contrast associated with the stimulus, motion contained in the stimulus, and a length of the stimulus.

4. The computer-implemented method of claim 1, further comprising halting, responsive to determining that the second objective function is not equivalent to the threshold minimum value, consideration of the second objective function.

5. The computer-implemented method of claim 4, further comprising utilizing, in the subsequent iteration, only the first objective function.

6. The computer-implemented method of claim 1, further comprising selecting, responsive to determining that the first objective function and the second objective function have not increased over a predetermined number of consecutive iterations, optimal stimulus parameters for the stimulus.

7. The computer-implemented method of claim 1, wherein the source localization technique is Low-Resolution Electromagnetic Tomography Analysis (LORETA).

8. The computer-implemented method of claim 1, wherein the first subset of the one or more voxel locations are grouped together based on identified roles in hierarchical sensory information processing.

9. The computer-implemented method of claim 1, wherein the second subset of the one or more voxel locations are grouped together based on spatial functional characteristics.

10. The computer-implemented method of claim 1, wherein the second subset of the one or more voxel locations correspond to one or more regions selected from the group consisting of: a brain frontal area, a centrotemporal area, a motor cortex, and a posterior parietal lobe.

11. A system for performing iterative adjustments to sensory stimuli, the system comprising:
one or more processors;
one or more computer readable media storing instructions that are executable by the one or more processors to perform operations for:
receiving brain activity data associated with a stimulus presented to a subject;
preprocessing the brain activity data;
performing a source localization technique on the preprocessed brain activity data to identify one or more voxel locations where electrical activity contained in the brain activity data is projected to originate;
grouping, subsequent to performance of the source localization technique and based on the preprocessed brain activity data, a first subset of the one or more voxel locations together, wherein the first subset includes is associated with sensory areas of interest;
grouping, subsequent to performance of the source localization technique and based on the preprocessed brain activity data, a second subset of the one or more voxel locations together, wherein the second subset is associated with non-sensory areas of interest;
determining a first objective function based on localized neural activity identified within the first subset of the one or more voxel locations and determining a second objective function based on the localized neural activity identified within the second subset of the one or more voxel locations;
determining whether the second objective function is equivalent to or exceeds a threshold minimum value;
averaging, responsive to determining that the second objective function is equivalent to or exceeds the threshold minimum value, an amount of the second objective function over the threshold minimum value with the first objective function to produce an averaged objective function; and
utilizing the averaged objective function to refine one or more parameters of the stimulus in a subsequent iteration of a closed-loop optimization function of the system.

12. The system of claim 11, wherein the stimulus is a visual stimulus.

13. The system of claim 11, wherein the one or more parameters of the stimulus include one or more of: a color associated with the stimulus, a contrast associated with the stimulus, motion contained in the stimulus, and a length of the stimulus.

14. The system of claim 11, wherein the instructions are executable by the one or more processors to further perform operations for:
halting, responsive to determining that the second objective function is not equivalent to the threshold minimum value, consideration of the second objective function.

15. The system of claim 11, wherein the instructions are executable by the one or more processors to further perform operations for:
selecting, responsive to determining that the first objective function and the second objective function have not increased over a predetermined number of consecutive iterations, optimal stimulus parameters for the stimulus.

16. The system of claim 11, wherein the source localization technique is Low-Resolution Electromagnetic Tomography Analysis (LORETA).

17. The system of claim 11, wherein the first subset of the one or more voxel locations are grouped together based on identified roles in hierarchical sensory information processing.

18. The system of claim 11, wherein the second subset of the one or more voxel locations are grouped together based on spatial functional characteristics.

19. The system of claim 11, wherein the second subset of the one or more voxel locations correspond to one or more regions selected from the group consisting of: a brain frontal area, a centrotemporal area, a motor cortex, and a posterior parietal lobe.

20. A non-transitory computer-readable medium storing computer-executable instructions which, when executed by a system, cause the system to perform operations comprising:
receiving, at a computing device associated with the system, brain activity data associated with a stimulus presented to a subject;
preprocessing, using a processor associated with the computing device, the brain activity data;
performing, using the processor, a source localization technique on the preprocessed brain activity data to identify one or more voxel locations where electrical activity contained in the brain activity data is projected to originate;
grouping, subsequent to performance of the source localization technique and based on the preprocessed brain activity data, a first subset of the one or more voxel locations together, wherein the first subset includes is associated with sensory areas of interest;
grouping, subsequent to performance of the source localization technique and based on the preprocessed brain activity data, a second subset of the one or more voxel locations together, wherein the second subset is associated with non-sensory areas of interest;
determining, using the processor, a first objective function based on localized neural activity identified within the first subset of the one or more voxel locations and determining a second objective function based on the localized neural activity identified within the second subset of the one or more voxel locations;
determining, using the processor, whether the second objective function is equivalent to or exceeds a threshold minimum value;
averaging, responsive to determining that the second objective function is equivalent to or exceeds the threshold minimum value, an amount of the second objective function over the threshold minimum value with the first objective function to produce an averaged objective function; and
utilizing the averaged objective function to refine one or more parameters of the stimulus in a subsequent iteration of a closed-loop optimization system.
